(19) United States
(12) Patent Application Publication
(10) Pub. No.: US 2003/0175224 A1
(43) Pub. Date: Sep. 18, 2003

(54) Method and material for applying a semipermanent marking stain to a portion of a person's body

(76) Inventor: Douglas C. Arndt, Ventura, CA (US)

Correspondence Address: Harold L. Jackson, Jackson Law Corporation, 14751 Plaza Dr., St. N, Tustin, CA, 92780, US

(21) Appl. No.: 10/238,068

(22) Filed: Sep. 9, 2002

Related U.S. Application Data
(63) Continuation of application No. 09/648,437, filed on Aug. 25, 2000, now abandoned.

(51) Int. Cl. A61K 7/04; A61K 7/021

(52) U.S. Cl. 424/61; 424/63

(57) ABSTRACT

A method for making a semipermanent mark on an individuals body comprises preparing a marking solution containing ninhydrin, preferably by mixing crystalline ninhydrin in water to provide a solution having a ninhydrin concentration of about 2% to 3% by weight and applying the resulting solution to a portion of the body such as a finger or thumb. A small quantity of a compatible dye, such as rhodamine, may also be added to the marking solution to produce an immediate, though less permanent, stain.

### FIELD OF THE INVENTION

The present invention relates to a reagent solution for interacting with organic materials present on the surface of human skin to provide a semipermanent marking stain for identification purposes and method for applying the same.

### BACKGROUND OF THE INVENTION

Many governments around the world and agencies thereof strive to deter duplicate voting during elections by requiring those casting ballots to immerse a digit such as a finger or thumb into a container of an indelible ink immediately prior to or after voting. The resulting stain enables those monitoring the voting process to identify a person as having previously voted and prevent the person from casting additional votes. Since the voting process in such governments often spans several days it is necessary that the stain remain visible for that period of time and not be readily removable through the use of common soaps, solvents and cleaners.

Dyes alone have proven to be unsatisfactory because they fail to remain on the skin for the required time. To improve the indelibility of such dyes, silver nitrate has been added to the staining solution. This reagent produces a brownish black stain that remains on the skin for greater than 48 hours. However, silver nitrate has several drawbacks. First, it is highly poisonous. Second, it has a short shelf life in that it is generally reduced to elemental silver by the dye used over a short period of time. Third, the stain color blends with dark skin and may be difficult to detect in many cases.

I have discovered a reagent solution which overcomes the above problems. The solution interacts with organic components which commonly reside on the surface of the skin to produce a semipermanent highly visible stain on a selected portion of a person's body. The key ingredient of the solution is ninhydrin which does not have the toxicity problem characteristic of silver nitrate. While ninhydrin has been used to develop latent fingerprints, to my knowledge, it has not been used to provide an identifying mark on a person.

### SUMMARY OF THE INVENTION

A method of making a semipermanent identifying mark on a portion of an individuals's body in accordance with the present invention comprises the steps of providing a solution of ninhydrin, preferably using water as the solvent and applying the solution to the selected body portion, such as a finger or thumb.

In addition to ninhydrin the solution also preferably contains a dye which is compatible with ninhydrin and is capable of producing a substantially immediately visible stain on the selected body portion.

### DESCRIPTION OF THE PREFERRED EMBODIMENT

The method of the present invention applies a reagent solution containing ninhydrin (triketohydrindene hydrate) and appropriate solvent such as water to a selected visible portion (e.g., finger or thumb) of the body of a person such as a voter to provide a semipermanent stain. Ninhydrin reacts with organic materials, such as free amino proteins and carboxyl groups present on the skin (and/or finger nails) of the person to produce a reaction product blue to purplish in color. The reaction takes about 5 to 10 minutes and the resulting stain is semipermanent in that it cannot be readily, removed through the use of common household cleaners and solvents. The stain generally remains visible for several days and disappears as the surface layer of the skin on which it resides is removed through the process of exfoliation.

The solution may be applied by immersing the recipient's finger or thumb into a suitable container of the solution or by applying the solution via a brush, etc. To satisfy the person, overseeing the marking process, that the recipient has indeed been exposed to the marking solution, it is preferable to add to the solution a dye which is compatible with ninhyprin and capable of producing a substantially immediate less permanent stain on the finger etc. While there are numerous dyes suitable for this purpose, I have found that rhodamine produces a magenta colored stain almost immediately after contact with the skin.

I have found that an acceptable staining solution may be made up with five (5) gallons of water to which is added about 450 grams of ninhydrin, in crystalline form, and about 45 grams of a dye such as rhodanine resulting in a ninhydrin concentration of about 2.3%. While the percentage of ninhydrin is not critical the cost of the resulting solution dictates that the percentage thereof be kept reasonably low, e.g., within the range of about 0.1% to 5% of the total solution by weight. By the same token the percentage of rhodanine in the solution is also not critical but is preferably within the range of 0.01% to 0.5% by weight.

There has thus been described a semipermanent marking stain which has a long shelf life, is not readily removable and remains on the recipient for several days enabling those monitoring an event such as voting to readily identify those that have previously participated in the event.

What is claimed is:

1. A method of making a,semipermanent identifying mark on a portion of a individual's body comprising:
a) providing a solution containing ninhydrin; and
b) applying the solution to a portion of the individual's body.

2. The method of claim 1 wherein the applying step comprises applying the solution to the tip of the individual's finger.

3. The method of claim 1 wherein the providing step includes dissolving ninhydrin in water.

4. The method of claim 3 wherein ninhydrin comprises about 0.1% to 5% of the solution by weight.

5. The method of claim 3 wherein the solution further includes a dye compatible with ninhydrin for providing an initial stain on said portion of the individual's body.

6. The method of claim 5 wherein the dye is rhodamine comprising about 0.1% to 5% of the solution by weight.

7. A reagent solution for providing a semipermanent mark on a portion of a person's body for identification purposes comprising a solution of ninhydrin, a dye compatible with ninhydrin and capable of providing a substantially immediately visible stain on the person's body and water with the ninhydrin and dye each comprising about 0.1% to 5% of the solution by weight.

8. The solution of claim 7 wherein the dye is rhodamine.

9. The solution of claim 7 wherein the ninhydrin comprises about 0.1% to 0.5% of the solution by weight.

10. The solution of claim 9 wherein the dye comprises about 0.01% to 0.5% of the solution by weight.

11. A method of providing a substantially insoluble and indelible mark on an individual's body part comprising:
a) mixing ninhydrin and a compatible dye in water to form a solution in which the ninhydrin and dye each comprise less than 5% of the solution by weight; and
b) applying the solution to said body part.
